(19) United States
(12) Patent Application Publication
(10) Pub. No.: US 2004/0047808 A1
(43) Pub. Date: Mar. 11, 2004

(54) Cystic fibrosis transmembrane-conductance regulator (cftr)-membrane translocation sequence fusion protein (cftr-mts) as a therapeutic agent

(76) Inventors: Arlene Stecenko, Atlanta, GA (US); Kenneth Birgham, Atlanta, GA (US); Mauricio Rojas, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 10/416,285

(22) PCT Filed: Nov. 9, 2001
(86) PCT No.: PCT/US01/49958

(51) Int. Cl. A61K 38/17; C07H 21/04; A61L 9/04; C12P 21/04; C07K 14/705

(52) U.S. Cl. 424/45; 514/12; 435/69.7; 435/320.1; 435/325; 530/350; 536/23.5

(57) ABSTRACT

The present invention provides a method of treating cystic fibrosis in a human subject diagnosed with cystic fibrosis, comprising administering to the subject, in a pharmaceutically acceptable carrier, an effective amount of a fusion protein, comprising a cystic fibrosis transmembrane conductance regulator (CFTR) and a membrane translocation sequence (MST), whereby the fusion protein can be taken up by affected cells in the subject, thereby treating cystic fibrosis. The present invention also provides a fusion protein comprising a cystic fibrosis transmembrane conductance regulator and a membrane translocation sequence.

### CROSS-REFERENCE TO RELATED APPLICATIONS

This application claims priority to U.S. provisional application Serial No. 60/247,494 filed on Nov. 9, 2000. The 60/247,494 provisional patent application is herein incorporated by this reference in its entirety.

### STATEMENT OF FEDERALLY SPONSORED RESEARCH

This invention was made with government support under Grant No. P30 DK 54759 awarded by the Public Health Service, National Institutes of Health, NIDDK. The government has certain rights in the invention.

### BACKGROUND OF THE INVENTION

1. Field of the Invention

The present invention relates to the field of the therapy of cystic fibrosis in human subjects. In particular, the present invention relates to a method of treating cystic fibrosis comprising administering a fusion protein to affected cells of the subject.

2. Background

Cystic fibrosis (CF) is caused by mutations in the gene encoding the cystic fibrosis transmembrane conductance regulator (CFTR) which is a chloride channel expressed in epithelial tissues. The mutations in CF patients result in abnormally rapid intracellular degradation of the CFTR protein. This defect in intracellular CFTR protein results in abnormal chloride transfer across epithelial membranes, causing excessive thickening of the mucus lining the airways. The viscid mucus allows for invasion by Staphylococcus aureus, Hemophilus influenzae and Pseudomonas aeruginosa which provoke a vigorous and excessive neutrophilic inflammatory response that over years causes lung destruction manifested as bilateral bronchiectasis.

CF is the most common lethal inherited disease in Caucasians. Over 25,000 people in the US have CF and death occurs, on average, in the third decade of life due progressive lung disease. No curative therapy is available and current medications only slow the progression of the lung disease. Health care costs are tremendous in these patients: on average a typical patient with CF with take 40 to 50 pills per day and 3 to 5 different inhalers. Most require hospitalization on at least a yearly basis. This level of care often begins in early childhood and extends through the life of the patient. Presently, the only “curative” treatment for CF lung disease is lung transplant, a costly procedure (>$150,000) with a 5 year survival of under 70%.

The development of an effective means to deliver the CFTR protein to the lungs would in essence cure the pulmonary manifestations of CF, provided the protein could be given repetitively and maintain function. This would greatly improve the quality of life for these patients as well as decrease or eliminate the need for costly (and not totally effective) supportive/symptomatic treatment.

The present addresses these needs by providing a method of introducing a functional CFTR protein into affected cells of patients diagnosed with cystic fibrosis (CF).

### BRIEF DESCRIPTION OF THE DRAWINGS OF THE INVENTION

FIG. 1A is a fluorescent micrograph of an indirect immuno-fluorescence assay (IIFA) of cells contacted with unmodified FGF-R.

FIG. 1B is a fluorescent micrograph of an indirect immuno-fluorescence assay (IIFA) showing the insertion of FGF-R-MTS fusion protein on the cell surface.

### DETAILED DESCRIPTION OF THE INVENTION

As used in the specification and the appended claims, the singular forms “a,” “an,” and “the” include plural referents unless the context clearly dictates otherwise. Thus, for example, reference to “a nucleic acid” includes multiple copies of the nucleic acid and can also include more than one particular species of nucleic acid molecule:

The present invention provides a fusion protein comprising a cystic fibrosis transmembrane conductance regulator (CFTR) and a membrane translocation sequence (MTS). The fusion protein can further comprise a tag, such as a His tag or GST among others. The MTS can be either C-terminal or N-terminal to the CFTR.

The CFTR of the present fusion protein comprises, for example, the entire normal human CFTR sequence. The term “CFTR” as used herein can include any domain of the normal CFTR protein having the chloride channel activity of the normal CFTR, including additions, substitutions and/or deletions in amino acids that retain the activity. Examples of other CFTRs include those described below and in the cited publications, which have been incorporated by reference.

The fusion protein also comprises a membrane translocation sequence (MTS), comprising, for example, the amino acid sequence AAVLLPVLLAAP (SEQ ID NO:1). The MTS can be a sequence of amino acids generally of a length of about 10 to about 50 or more amino acid residues, many (typically about 55-60%) residues of which are hydrophobic such that they have a hydrophobic, lipid-soluble portion. The hydrophobic portion is a common, major motif of the signal peptide, and it is often a central part of the signal peptide of protein secreted from cells. A signal peptide is a peptide capable of penetrating through the cell membrane to allow the export of cellular proteins. The signal peptides of this invention, are also MTSs, i.e., capable of penetrating through the cell membrane from outside the cell to the interior of the cell. The amino acid residues can be mutated and/or modified (i.e., to form mimetics) so long as the modifications do not affect the translocation-mediating function of the peptide. Thus the word “peptide” includes mimetics and the word “amino acid” includes modified amino acids, as used herein, unusual amino acids, and D-form amino acids. All MTS peptides encompassed by this invention have the function of mediating translocation across a cell membrane from outside the cell to the interior of the cell. Such MTS peptides could potentially be modified such that they lose the ability to export a protein but maintain the ability to import molecules into the cell. A putative signal peptide can easily be tested for this importation activity following the teachings provided herein, including testing for specificity for any selected cell type.

A detailed description of the MTS of the present invention and its use in conjunction with biologically active proteins and peptides is presented in U.S. Pat. No. 6,248,558, and PCT Publication WO 01/37821, which are incorporated herein in their entireties, and specifically for their teaching of the MTS (also referred to as importation competent signal peptides) and for the making of MTS fusion proteins.

Thus the fusion proteins of the invention include the following: CFTR-MTS, GST-CFTR-MTS and HIS-CFTR-MTS. Additional fusion proteins taught herein include FGFR-MTS, GST-FGFR-MTS and HIS-FGFR-MTS.

The present invention also provides nucleic acids which encodes fusion proteins of the invention, more specifically the fusion proteins comprising CFIR-MTS. “Nucleic acid” as used herein includes single- or double-stranded molecules which may be DNA, comprised of the nucleotide bases A, T, C and G, or RNA, comprised of the bases A, U (substitutes for T), C, and G. The nucleic acid may represent a coding strand or its complement. Nucleic acids may be identical in sequence to the portion of the sequence which is naturally occurring or may include alternative codons which encode the same amino acid as that which is found in the naturally occurring sequence. Furthermore, nucleic acids can include codons which represent conservative substitutions of amino acids as are well known in the art.

Also provided are sequences which selectively hybridize with the recombinant nucleic acids encoding the CFTR-MTS fusion protein. For example, a nucleic acid can selectively hybridize under stringent conditions. “Stringent conditions” refers to the hybridization conditions used in a hybridization protocol or in the primer/template hybridization in a PCR reaction. In general, these conditions should be a combination of temperature and salt concentration for washing chosen so that the denaturation temperature is approximately 5-20° C. below the calculated Tₘ (melting/denaturation temperature) of the hybrid under study. The temperature and salt conditions are readily determined empirically in preliminary experiments in which samples of reference nucleic acid are hybridized to the primer nucleic acid of interest and then amplified under conditions of different stringencies. The stringency conditions are readily tested and the parameters altered are readily apparent to one skilled in the art. For example, MgCl₂ concentrations used in PCR buffer can be altered to increase the specificity with which the primer binds to the template, but the concentration range of this compound used in hybridization reactions is narrow, and therefore, the proper stringency level is easily determined. For example, hybridizations with oligonucleotide probes 18 nucleotides in length can be done at 5-10° C. below the estimated Tₘ in 6× SSPE, then washed at the same temperature in 2× SSPE. The Tₘ of such an oligonucleotide can be estimated by allowing 2° C. for each A or T nucleotide, and 4° C. for each G or C. An 18 nucleotide probe of 50% G+C would, therefore, have an approximate Tₘ of 54° C. Likewise, the starting salt concentration of an 18 nucleotide primer or probe would be about 100-200 mM. Thus, stringent conditions for such an 18 nucleotide primer or probe would be a Tₘ of about 54° C. and a starting salt concentration of about 150 mM and modified accordingly by preliminary experiments. Tₘ values can also be calculated for a variety of conditions utilizing commercially available computer software (e.g., OLIGO®).

The nucleic acid encoding a protein of this invention can be part of a recombinant nucleic acid construct comprising any combination of restriction sites and/or functional elements as are well known in the art which facilitate molecular cloning and other recombinant DNA manipulations. Thus, the present invention further provides a recombinant nucleic acid construct comprising a nucleic acid encoding a protein of this invention.

The nucleic acid encoding a protein of this invention can be any nucleic acid that functionally encodes a protein of this invention. To functionally encode a protein (i.e., allow the nucleic acids to be expressed), a nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Preferred expression control sequences are promoters derived from metallothionine genes, actin genes, immunoglobulin genes, CMV, SV40, adenovirus, bovine papilloma virus, etc. A nucleic acid encoding a selected protein can readily be determined based upon the genetic code for the amino acid sequence of a selected protein and many nucleic acids will encode any selected protein. Modifications in the nucleic acid sequence encoding a protein are also contemplated. Modifications that can be useful are modifications to the sequences controlling expression of a protein to make production of a protein inducible or repressible as controlled by the appropriate inducer or repressor. Such methods are standard in the art. The nucleic acid of this invention can be generated by means standard in the art, such as by recombinant nucleic acid techniques and by synthetic nucleic acid synthesis or in vitro enzymatic synthesis.

Moreover, the present invention provides a vector comprising the nucleic acid which encodes a CFTR-MTS fusion protein. The vector can be an expression vector which contains all of the genetic components required for expression of the nucleic acid in cells into which the vector has been introduced, as are well known in the art. The expression vector can be a commercial expression vector or it can be constructed in the laboratory according to standard molecular biology protocols. Specific vectors and expression systems that exemplify the types of vectors and expression systems that can be used to express the CFTR-MTS fusion proteins are provided in the Examples.

A fusion protein of the present invention can be in a pharmaceutically acceptable carrier. By “pharmaceutically acceptable” is meant a material that is not biologically or otherwise undesirable, i.e., the material may be administered to an individual along with the a protein without causing substantial deleterious biological effects or interacting in a deleterious manner with any of the other components of the composition in which it is contained.

The present invention also provides a method of treating cystic fibrosis in a human subject diagnosed with cystic fibrosis comprising administering to the subject, in a pharmaceutically acceptable carrier, an effective amount of the fusion protein of the present invention, whereby the fusion protein can be taken up by affected cells in the subject.

The principal route of administration of the CFTR/MTS fusion protein is by aerosol in order to deliver the protein to the lung airway epithelium. However, because patients with cystic fibrosis also have dysfunction of the gastrointestinal tract and of the liver, other routes of administration, for example, intravenous, intramuscular, and intraperitoneal delivery and infusion into the afferent blood supply to the liver. Oral administration can also be used in view of the availability of methods to protect the protein from gastric secretions, e.g., by microencapsulation, etc.

The exact amount of the protein required will vary from subject to subject, depending on the age, weight and general condition of the subject, the particular protein used, its mode of administration and the like. Thus, it is not possible to specify an exact amount for every protein. However, an appropriate amount can be determined by one of ordinary skill in the art using only routine experimentation. For example, a dose of about 1 μg to about 100 μg of the protein can be administered in an aerosol to the lungs of a subject. The amount of protein administered can be adapted to the specific mode of administration by the skilled clinician. The protein can be administered from about once per week to about 3 times per day.

The present invention is more particularly described in the following examples which are intended as illustrative only since numerous modifications and variations therein will be apparent to those skilled in the art.

### EXAMPLES

Production of a CFTR-MTS Fusion Protein.

A membrane translocation sequence can be made as described in U.S. Pat. No. 5,807,746 (incorporated herein in its entirety); PCT Publication WO 01/37821 (incorporated herein in its entirety); in “Regulation of NF-κB, AP-1, NFAT, and STAT 1 Nuclear Import in T Lymphocytes by Noninvasive Delivery of Peptide Carrying the Nuclear Localization Sequence of NF-κB p50^{I},” Torgerson et al., The Journal of Immunology, pages 6084-6092, 1998 (incorporated herein in its entirety); and “Genetic Engineering of Proteins with Cell Membrane Permeability,” Rojas et al., Nature Biotechnology, Volume 16, pages 370-375, April, 1998 (incorporated herein in its entirety). As a specific example, the MTS used in this procedure has the following sequence AAVLLPVLLAAP (SEQ ID NO:1).

The CFTR gene is known (Riordan et al., 1989, Science 245:1066-1073; Rommens et al., 1989, Science 245: 1059-1065).

Generation HIS-MTS cDNA Clone

Due to the size of the CFTR cDNA clone we designed a new MTS vector and used a double vector expression system. The present vector expresses the MTS fusion protein associated to the HIS tag sequence instead of GST. As the first step, the CFIR cDNA was introduced into the QIA-expressionist (pQE) from Qiagen, a commercially available vector that contains the HS-tag. The cDNA was introduced into the pQE vector, which permits the expression of a high level of His-tag fusion proteins under the control of lac Z. A second vector, pREP4, is used to regulate the expression of the fusion proteins in the pQE vector. The pREP4 vector constitutively express the lac repressor, and its function can be inhibited by the addition of IPTG which stimulates the expression of pBR22 (represses the repressor). Once the repressor is inactivated the bacteria begin the transcription of the fusion proteins. The bacteria can be kept at refrigeration temperature for several hours to allow for better folding of the expressed fusion proteins.

The primers designed to introduce MTS into the pQE vectors were:

| | |
|---|---|
| Primer pQEPst-mts1 (50-mer) | |
| TGC AGC CCC GCA GCC GTT CTT CTC CCT GTT CTT CTT GCC GCA CCC TAA GG; and | (SEQ ID NO:2) |
| Primer pQEPst-mts2 (50-mer) | |
| TGC ACC TTA GGG TGC GGC AAG AAG AAC AGG GAG AAG AAC GGC TGC GGG GC. | (SEQ ID NO:3) |

Generation His-CFTR and His-CFTR-MTS Fusion Proteins

The βA-CFTR-BQ plasmid was obtained from John F. Engelhard of Iowa University, and is a 7762 pb plasmid containing the complete cDNA of CFTR. Other plasmids containing the cDNA of CFTR can be obtained or routinely generated. Pst1 digestion released the DNA insert that corresponds to CFTR. After Pst1 digestion, the CFTR fragment was gel purified following the protocol of QIAGEN QIAEX II gel extraction kit. The purified fragment was ligated into Pst1 digested pQE32 and pQE32-MTS vectors (from QIAGEN) and introduced into a bacteria strain containing pREP4 plasmid.

Selection of Positive Clones by In-Situ Hybridization

An oligonucleotide probe was designed from the C-terminal sequence of CFTR cDNA. This probe was label by T4 kinase using γ³²P, and used to screen CFTR-pQE32 and CFTR-pQE32-MTS transformations. Approximately 400 colonies were screened and 32 positive colonies were identified.

After ON culture with IPTG, protein expression was induced in these clones and total cell lysates were used in a SDS-PAGE gel. Coomasie staining of SDS-PAGE gels identified 8 different clones that express a protein with the expected molecular size for HIS-CFTR/HIS-CFTR-MTS. In addition, fusion protein was identified by western blot analysis using an anti-HIS and a CFTR antibodies. Six clones were positive for the expression of HIS-CFTR fusion protein (about 160 kDa). The fusion protein produced was HIS tagged CFTR with MTS at the C-terminus.

This procedure can be modified using procedures set forth in Sambrook et al., Molecular Cloning: A Laboratory Manual, 2nd Ed., Cold Spring Harbor Laboratory, Cold Spring Harbor, N.Y., 1989.

Generation of FGFR-MTS

Introduction of BamH1 restriction sites into the FGFR to clone

PCR primers:
FGFR1ba1-1

(SEQ ID NO:4)

CCGGATCCCCATGTGGAGGTGGAAGAGCGTCGTCTTCTGG; and
FGFR1ba1-2

(SEQ ID NO:5)

CCGGATCCCGCGGCGTTTGAGTCCCCGATTGGCAAGCTG

| PCR protocol: | | |
|---|---|---|
| 10xTaq Buffer (Perkin Elmer) | 5.0 | μl |
| MgCl₂ 25 mM | 4.0 | μl |
| Primer bal-1 10 μM | 2.5 | μl |
| Primer bal-2 10 μM | 2.5 | μl |
| cDNA FGFR1 (in pCDNA3.1) | 10.0 | μl |
| from G. Miller 1 ng/ul | | |
| dNTP 2.5 mM each | 8.0 | μl |
| Taq DNA polymerase | 1.0 | μl |
| H₂O | 16.0 | μl |

| | |
|---|---|
| PCR cycle: | |
| 95° C. 1 min | ×35 cycles |
| 65° C. 2 min | |
| 72° C. 2 min | |

The PCR product was purified by gel extraction using QIAGEN QIAEX II kit following the protocol recommended by the manufacture. After purification, the PCR product was inserted into pGEMT-easy (Promega). This vector is modified to insert PCR products.

MTS-2 Cloning

FGFR1 cDNA was released from the pGEM-T by BamH1 digestion and subcloned into MTS-2 vector (pGEX-3×expression vector (Promega) containing MTS sequence). The FGFR1-MTS-2 construct was introduced into BL-21 bacteria strain (high efficiency in protein expression) and positive clones were determined by isolation of the plasmids and digestion with different restriction enzymes

Import Assays and Western Blot

2×10⁶ BaF3 (T cells without FGFR) cells were incubated with 35 μg/ml of the fusion proteins: GST, GST-FGFR1 and GST-FGFR1-MTS, during 2 hours at 37° C. Cells were washed four times with cold-PBS, transferred to a new tube and washed one time more. The pellet was resuspended in SDS-loading buffer and 20 μl were used in a 7.5% SDS-PAGE. The presence of the fusion protein was detected using an anti-GST and anti-FGFR antibodies. Protein was detected only in the samples in which the cells were incubated with GST-FGFR1-MTS.

Indirect Immuno-Fluorescence Assay (IIFA)

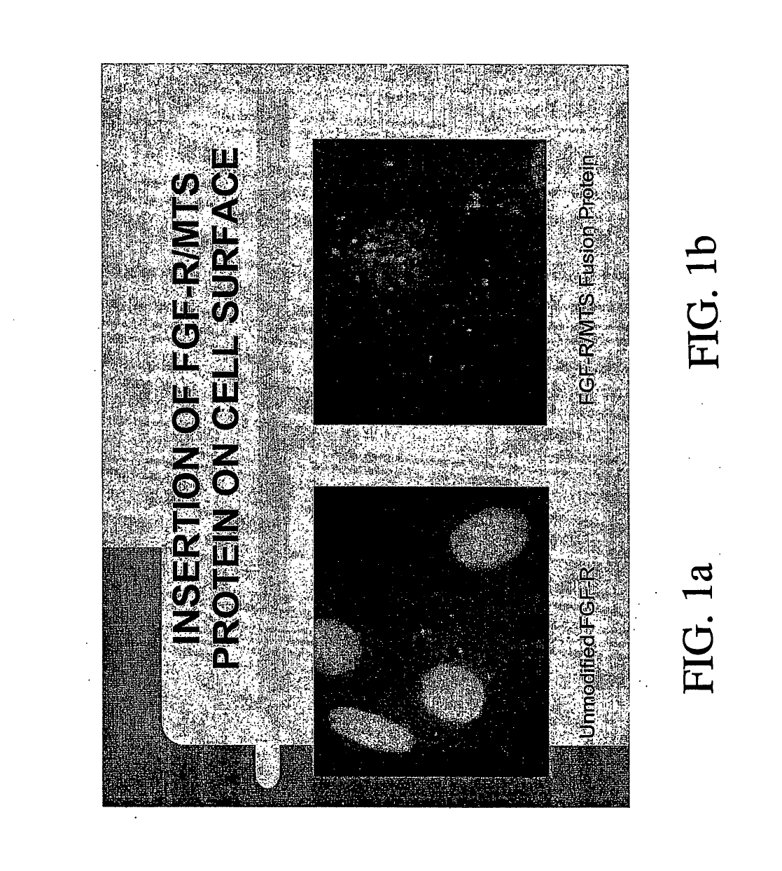

We used IIFA to demonstrate the membrane localization of GST-FGFR1-MTS fusion protein. Briefly, NIH3T3 cells were cultured in 4 well-chamber slides (Nunc, CA) and grown for 3 days at 37° C. Sub-confluent cells were washed with serum-free medium and incubated with the GST-FGFR1 and GST-FGFR1-MTS proteins at a concentration of 10 μg/ml during 1 hour at 37° C. The cells were washed with cold PBS and fixed with 3.7% paraformaldehyde in PBS at RT during 15 min. Cells were washed with PBS, and treated or not with 0.25% Triton X-100 for 10 min to permeabilize the cell membrane. Cells were incubated with blocking solution (PBS+1% goat serum +1% BSA) for 30 min at 37° C. After blocking, cells were incubated with anti-GST in PBS+1% BSA for 2 hours. Cells were washed 3 times with PBS and blocked for 30 min. Protein-antibody complexes were incubated during 1 hour with goat anti-rabbit IgG labeled with Texas Red (Southern Biotech, AL). Cells were washed 3 times. Nuclei were stained with DAPI, diluted in 2% SSC (1:1000) and incubated for 1 min at RT Molecular Probes, OR). Cells were washed 3 times with PBS. Coverslips with stained cells were mounted in ProLong Antifade (Molecular Probes, OR) and analyzed in a fluorescence microscope. Fluorescence microscopy identified the fusion protein in the outer cell membrane, the normal site for this receptor. Functional studies confirmed presence of a functioning FGF receptor (see FIGS. 1A and 1B).

Functional CFTR Assay: Patch Clamp Assay

The CFTR protein is a transmembrane cyclic AMP (cAMP) activated chloride channel, and one of the standard means to assess appropriate functioning of this channel is through the patch clamp assay. The delivery of an active CFTR-MTS fusion protein into cells is measured using a patch clamp assay on cells to which the fusion protein has been administered.

Briefly, whole-cell currents are measured in the whole-cell configuration of the patch clamp technique (Hamill et al. Nature 294(5840):462-464, 1981, which is incorporated by reference for the teaching of the patch clamp technique), using an Axopatch 200A amplifier (Axon Instruments, Inc). The compositions of the bath and pipette solutions are designed with Cl⁻ as the main conducting ion. The control bath solution contains (in mM): 140 NMDG-Cl (N-methyl-D-glucamine-chloride), 2.0 CaCl₂, 2.0 MgCl₂, 5 HEPES (N-[2-hydroxyethyl]piperazine-N′-[2-ethanosulfonic acid], pH 7.4, ˜275 mosmol/kg. The pipette solution contains (in mM): 140 NMDG-Cl, 2.0 MgCl₂, 5 HEPES, 5 MGATP, 1 EGTA (ethylene glycol-bis-[β-aminoethyl ether], pH 7.4, ˜270 mosmol/kg. The pipette solution is diluted 9-13% with distilled water to prevent activation of swelling-activated Cl⁻ currents. As a reference electrode, an agar-bridge with composition similar to the control bath solution are utilized. The holding potential is 0 mV in all experiments, and the whole-cell currents are measured from −100 to +100 mV (in 20 mV steps) 50 ms after the start of the voltage pulse. Pulse generation, data collection and analyzes are done with Clampex 7.0 (Axon Instruments, Inc.). The anionic selectivity of the cAMP-activated current is determined in NaCl bath solution (in mM): 140 NaCl, 2.0 CaCl₂, 2.0 MgCl₂, 5 HEPES, pH 7.4, ˜275 mosmol/kg) by replacing NaCl with equimolar NaBr, NaI, or Na-gluconate. The liquid-junction potentials generated upon changing the extracellular anion are calculated using the Junction Potential Calculator in Clampex 7.0.

After obtaining whole-cell, currents are monitored ≧8 min to allow for pipette and intracellular solutions to equilibrate. After this period, the cells are exposed to a cAMP cocktail (in μM, 100 IBMX, 100 8-Br-cAMP and 25 forskolin). Cells studied are immortalized CF airway epithelial cells as a negative control and tsA201 cells transfected with a plasmid encoding wild-type CFTR as a positive control. The CF cells, as expected, fail to exhibit cAMP-activated currents. In contrast, the human CFTR-transfected tsA201 cells exhibit a linear Cl⁻ current with an ionic selectivity characteristic of CFTR-induced currents. The onset of this current is within 1 minute of exposure to the cAMP cocktail and reached steady-state after 5 minutes of exposure.

Although the present process has been described with reference to specific details of certain embodiments thereof, it is not intended that such details should be regarded as limitations upon the scope of the invention except as and to the extent that they are included in the accompanying claims.

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A fusion protein comprising a cystic fibrosis transmembrane conductance regulator (CFTR) and a membrane translocation sequence (MTS).

2. The fusion protein of claim 1, wherein the cystic fibrosis transmembrane conductance regulator is the entire human CFTR sequence.

3. The fusion protein of claim 1, wherein the membrane translocation sequence comprises the amino acid sequence AAVLLPVLLAAP (SEQ ID NO:1).

4. The fusion protein of claim 1 in a pharmaceutically acceptable carrier.

5. A nucleic acid encoding the protein of claim 1.

6. The nucleic acid of claim 5 in a vector.

7. A method of treating cystic fibrosis in a human subject diagnosed with cystic fibrosis comprising administering to the subject, in a pharmaceutically acceptable carrier, an effective amount of the fusion protein of claim 1, whereby the fusion protein can be taken up by affected cells in the subject, thereby treating cystic fibrosis.

8. The method of claim 7, wherein the fusion protein is administered in an aerosol.


SEQUENCE LISTING (5 sequences)

SEQ ID NO: 1 - protein, 12 aa, Artificial Sequence
  Description of Artificial Sequence; Note = synthetic construct
AAVLLPVLLAAP

SEQ ID NO: 2 - DNA, 50 bp, Artificial Sequence
  Description of Artificial Sequence; Note = synthetic construct
tgcagccccgcagccgttcttctccctgttcttcttgccgcaccctaagg

SEQ ID NO: 3 - DNA, 50 bp, Artificial Sequence
  Description of Artificial Sequence; Note = synthetic construct
tgcaccttagggtgcggcaagaagaacagggagaagaacggctgcggggc

SEQ ID NO: 4 - DNA, 40 bp, Artificial Sequence
  Description of Artificial Sequence; Note = synthetic construct
ccggatccccatgtggagctggaagagcctcctcttctgg

SEQ ID NO: 5 - DNA, 39 bp, Artificial Sequence
  Description of Artificial Sequence; Note = synthetic construct
ccggatcccgcggcgtttgagtccccgattggcaagctg